(19) United States
(12) Patent Application Publication
(10) Pub. No.: US 2026/0243769 A1
(43) Pub. Date: Aug. 20, 2026

(54) METHOD FOR THE DETECTION OF LUNG CANCER

(71) Applicant: Belgian Volition SRL, Isnes (BE)

(72) Inventors: Marielle HERZOG, Isnes (BE); Julie Alberte Hélène Ghislaine CANDIRACCI, Isnes (BE); Léa Louise Lorence PAYEN, Lyon (FR); Sébastien Loïc Guillaume COURAUD, Lyon (FR)

(21) Appl. No.: 19/164,395

(22) PCT Filed: Mar. 13, 2024
(86) PCT No.: PCT/EP2024/056664

§ 371 Date: Sep. 11, 2025

(30) Foreign Application Priority Data
Mar. 13, 2023 (EP) 23305334.7

(51) Int. Cl. G01N 33/5752; C12Q 1/6886

(52) U.S. Cl. CPC G01N 33/5752; C12Q 1/6886; C12Q 2600/156; G01N 2440/12

(57) ABSTRACT

The invention relates to methods of analysing cancer comprising detecting or measuring the level of trimethylation of lysine 27 on histone H3 (H3K27Me3) of a cell-free nucleosome in a body fluid sample obtained from a subject, as well as related uses and kits using said method.

### FIELD OF THE INVENTION

The invention relates to a body fluid test method for the monitoring and detection of cancer, particularly but not exclusively lung cancer, using a biomarker. The invention finds particular use in monitoring lung cancer patients both during and after treatment.

### BACKGROUND OF THE INVENTION

Cancer is a common disease with a high mortality. The biology of the disease is understood to involve a progression from a pre-cancerous (initiation) state leading to stage I, II, III and eventually stage IV cancer. For the majority of cancer diseases, mortality varies greatly depending on whether the disease is detected at an early localized stage, when effective treatment options are available, or at a late stage when the disease may have spread within the organ affected or beyond when treatment is more difficult.

Late-stage cancer symptoms are varied including blood discharged with coughing and unexplained weight loss as well as many other possible symptoms depending on the cancer type. However, most cancers diagnosed due to such symptoms will already be late stage and difficult to treat. Most cancers are symptomless at early stage or present with non-specific symptoms that do not help diagnosis. Cancer should ideally therefore be detected early using cancer tests.

The cancer with the highest mortality rate in developed countries is lung cancer. The five-year survival rate for lung cancer is >50% for cases detected when the disease is still localized within the lungs, but only 5% when the disease has spread to other organs. Unfortunately, most lung cancer cases are diagnosed when already metastatic (57%) whilst only 16% are diagnosed at an early stage.

Despite recent advances, very few blood test methods are used routinely during cancer screening. There is a need to develop further non-invasive blood tests for individual cancers for cancer diagnosis in general, to rule cancer in or out as a potential diagnosis in symptomatic patients or as an adjunct to other cancer detection methods.

Similarly, once a patient has undergone treatment, for example, surgery or radiotherapy with curative intent, the patient needs to be monitored over a period of time for possible reoccurrence. Currently this may take the form of periodic CT scans, PET scans or X-rays. These scans are expensive and over time the risk of radiation damage increases. PET scans and CT scan with imaging dye are also invasive and the patient can have an adverse reaction to the radioactive imaging tracer or dye used in these scans. Such scans are commonly only carried out at a minimum of 3-month intervals both to reduce exposure to radiation and to allow a radiographer to track disease over a time period. It would be extremely useful to have a simple blood test which would allow a physician to monitor a patient's response to treatment—be it historical or ongoing treatment. This would allow for more frequent monitoring and be both cheaper and faster than carrying out a scan. Such a blood test could be used to inform whether further treatment is needed if e.g. the cancer has reoccurred or whether to change the line of treatment if the patient is not responding to current therapy.

The present invention solves the aforementioned needs.

### STATEMENTS OF THE INVENTION

According to a first aspect of the invention, there is provided a method of analysing cancer in a subject, comprising:
(i) detecting or measuring the level of trimethylation of lysine 27 on histone H3 (H3K27Me3) of a cell-free nucleosome in a body fluid sample obtained from the subject; and
(ii) analysing circulating tumour DNA (ctDNA) obtained from the subject for a tumour-associated mutation.

According to one aspect of the invention there is provided a method of analysing cancer in a subject, comprising:
detecting or measuring the level of trimethylation of lysine 27 on histone H3 (H3K27Me3) of a cell-free nucleosome in a body fluid sample obtained from the subject; and
using the level detected or measured in the body fluid sample to analyse the cancer. In one embodiment, the presence of ctDNA in combination with a higher level of H3K27Me3 compared to a control is indicative of the presence and/or progression of cancer, is indicative that the subject requires further investigation, such as a lung biopsy, and/or is indicative that the subject requires treatment or a change of treatment; or wherein the absence of ctDNA in combination with a higher level of H3K27Me3 compared to a control is indicative of the presence and/or progression of cancer, is indicative that the subject requires further investigation, such as a lung biopsy, and/or is indicative that the subject requires treatment or a change of treatment.

In one embodiment the cancer is lung cancer.

In one embodiment the lung cancer is non-small cell lung cancer (NSCLC) or small cell lung cancer (SCLC).

In one embodiment the lung cancer is NSCLC.

In one embodiment the NSCLC is adenocarcinoma, squamous cell cancer, large cell carcinoma, adenosquamous carcinoma or sarcomatoid carcinoma.

In one embodiment the NSCLC is adenocarcinoma.

In one embodiment the method of the present invention is for monitoring progression of cancer, monitoring treatment, assessing the requirement for treatment, assessing the requirement for a change in treatment, assessing the requirement for investigation in a subject having cancer or suspected of having cancer, and/or assisting in the diagnosis of a subject having or suspected of having cancer.

In one embodiment the method is for managing a treatment plan in a subject who is suspected of having cancer, has cancer or who has previously received a cancer diagnosis.

In one embodiment the method is for assessing relapse following treatment for cancer.

In one embodiment, the subject has minimal residual disease (MRD).

In one embodiment the method is for assessing the success of cancer treatment with curative intent.

In one embodiment the method is for assessing the requirement for treatment of a subject or a change in a subject's treatment.

In one embodiment of the method of the invention the level of H3K27Me3 is compared to a control or a previous level obtained from the subject.

In one embodiment of the method of the invention includes monitoring a change in stage of cancer, wherein an increase, relative to an earlier stage sample or control is indicative of progression of the cancer from an earlier stage to later stage of disease.

In one embodiment the subject is subsequently investigated for cancer and the investigation includes pulmonary function test (PFT), imaging, biopsy and/or surgery.

In one embodiment the investigation is a biopsy.

In one embodiment the imaging is an x-ray, a chest computed tomography (CT) scan, or a positron emission tomography (PET) scan.

In one embodiment the level of H3K27Me3 which is indicative of the presence and/or progression of cancer is greater than or equal to about 22.5 ng/ml.

In one embodiment the level of H3K27Me3 is measured as one of a panel of measurements including DNA analysis.

In one embodiment the cancer is stage I, II, IIIA and B or IV.

In one embodiment the cancer is described as TX, T0, Tis, T1 (including T1mi, T1a, T1b, T1c), T2 (including T2a, T2b), T3 or T4; wherein the cancer is described as NX, N0, N1, N2 or N3 and/or wherein the cancer is described as M0 or M1 (including M1a, M1b, M1c).

In a second aspect of the present invention there is provided a method of analysing cancer in a subject, comprising:
(i) detecting or measuring the level of trimethylation of lysine 27 on histone H3 (H3K27Me3) of a cell-free nucleosome in a body fluid sample obtained from the subject; and
(ii) analysing ctDNA obtained from the subject for a tumour-associated mutation such as a tumour-associated somatic mutation.

In a third aspect of the present invention there is provided use of H3K27Me3 as a biomarker in a body fluid sample for monitoring progression of cancer, monitoring treatment, assessing the requirement for treatment, assessing the requirement for a change in treatment, assessing the requirement for investigation in a subject having cancer or suspected of having cancer, or assisting in the diagnosis of a subject having or suspected of having cancer.

In a fourth aspect of the present invention there is provided a kit comprising reagents to detect H3K27Me3 and optionally one or more biomarkers including ctDNA.

### SUMMARY OF THE INVENTION

The present invention relates to circulating H3K27 nucleosomes to monitor lung cancer patients during treatment—a universal biomarker quantifying the minimal residual disease (MRD) in plasma samples.

Treatment options of lung cancer comprise radiotherapy, and/or combined treatment approaches, including chemotherapy, immunotherapy and targeted therapies based on the tumoral molecular profile. Following curative-intent first-line therapies, clinical surveillance involves serial CT imaging. However, such surveillance can detect only macroscopic disease recurrence and is frequently inconclusive. Next Generation Sequencing (NGS) has been utilized to help identify and monitor treatment plans. Nucleosomes, complexes of DNA and histones proteins, are released during cell death into blood circulation. Trimethylation of lysine 27 on histone H3 (H3K27Me3), catalyzed by enhancer of zeste homolog 2 (EZH2), is a crucial epigenetic process in tumorigenesis. We investigated if H3K27Me3− nucleosome concentration could be a biomarker for minimal residual disease (MRD).

High levels of H3K27Me3 could allow physicians to detect MRD in lung cancer patients following treatment with curative intent. This could be achieved by monitoring testing of patients at defined intervals of treatment and recovery, alongside imaging, to incorporate analyses of evolving molecular landscapes during treatment. In this setting, the H3K27Me3− nucleosome quantification, to complete the molecular exploration of cfDNA is highly encouraging, especially in advanced NSCLC, where re-tissue biopsies are impractical, expensive, and may cause undue harm. H3K27Me3− nucleosome quantification may also be useful in patient identification for specific treatments such as EZH2 inhibitor.

The present invention also relates to circulating H3K27 Methylated Nucleosome concentration in lung cancer improves the contributive value of ctDNA molecular profiling result at diagnosis.

In patients with advanced lung adenocarcinoma cancers, a subset can be cured by radiotherapy, and/or combined regimens, including chemotherapy, immunotherapy, or targeted therapies based on somatic molecular profiling. As described above, nucleosomes are basic elements of chromatin, composed of 147 bp DNA wrapped around an octamer of histones. Cell-free DNA (cfDNA) and nucleosomes are released into the bloodstream upon cell death. In addition to genetic somatic alterations, epigenetic modifications are found to play a key role in tumorigenesis of different cancers. The trimethylated histone H3 at lysine 27 (H3K27Me3) is well known as a transcription-repressive mark and proven to be involved in tumorigenesis, cell cycle progression and proliferation dysregulation. However, the concentration of circulating nucleosomes, as a biomarker of the contributive value of circulating tumor DNA (ctDNA) molecular profiling in patient management at diagnosis has not previously been investigated.

High levels of H3K27Me3 and the absence of detecting somatic alterations strongly support the presence of non-mutated ctDNA in the corresponding plasma. This greatly improves the confidence in the negative molecular results in cfDNA in lung cancer. This may allow the indication of individualized therapeutic regimens with a short-time medical decision and may reduce invasive tissue re-biopsies.

### BRIEF DESCRIPTION OF THE FIGURES

FIG. 1 is a diagram showing an example of a current patient pathway during NSCLC progression after or during treatment. The pathway is based on presence/absence of somatic alterations in circulating tumor DNA (ctDNA). Number of samples (n=) are presented for the whole cohort (W; n=304). Percentages express the part of the total cohort involved.

FIG. 2 is a diagram showing an example of a patient pathway during NSCLC progression after or during treatment according to the invention. The pathway is based on H3K27Me3− nucleosomes levels below or above 22.5 ng/mL and on presence/absence of somatic alterations in circulating tumor DNA (ctDNA). Number of samples (n=) are presented for the whole cohort (W; n=304). Percentages express the part of the total cohort involved.

FIG. 3 is a diagram showing an example of a patient pathway at diagnosis according to the invention. The pathway is based on H 3K27Me3− nucleosomes levels below or above 22.5 ng/ml and on presence/absence of somatic alterations in circulating tumor DNA (ctDNA). Number of samples (n=) are presented for the whole cohort (W; n=318). Percentages express the part of the total cohort involved.

FIG. 4 shows quantification of circulating H3K27Me3− nucleosomes in NSCLC and healthy samples in the global cohorts at diagnosis. (A) Box plot analyses of circulating H3K27Me3− nucleosome levels in NSCLC samples compared with healthy samples. (B) Box plot analyses of level of circulating H3K27Me3− nucleosomes in NSCLC samples when mutated cfDNA was detected in blood (ctDNA+) compared to samples from NSCLC in which no mutation is detected (ctDNA−). The boxes represent 25^{th}-75^{th} percentile with median. Whiskers represent 2.5^{th}-97.5^{th} percentile. *** and **** represent p-value <0.001 and <0.0001 respectively, calculated by Mann-Whitney (groups of interest (k)=2) and Kruskal-Wallis (k>2) tests. (C) Receiver-operating characteristic (ROC) curve analysis of circulating H3K27Me3− nucleosomes. The straight dotted line indicates the theoretical random chance.

FIG. 5 shows quantification of circulating H3K27Me3− nucleosomes in NSCLC and healthy samples in the global cohorts during progression. (A) Box plot analyses of circulating H3K27Me3− nucleosome levels in NSCLC samples compared with healthy samples. (B) Box plot analyses of level of circulating H3K27Me3− nucleosomes in NSCLC samples when mutated cfDNA was detected in blood (ctDNA+) compared to samples from NSCLC in which no mutation is detected (ctDNA−). The boxes represent 25^{th}-75^{th} percentile with median. Whiskers represent 2.5^{th}-97.5^{th} percentile. *** and **** represent p-value <0.001 and <0.0001 respectively, calculated by Mann-Whitney (groups of interest (k)=2) and Kruskal-Wallis (k>2) tests. (C) Receiver-operating characteristic (ROC) curve analysis of circulating H3K27Me3− nucleosomes. The straight dotted line indicates the theoretical random chance. (D) The groups size, areas under the curve (AUC) and corresponding p-values for the targeted groups.

### DETAILED DESCRIPTION OF THE INVENTION

Despite recent advances, the diagnosis and treatment of cancer, particularly lung cancer, remains challenging. Currently, there is no circulating protein biomarker available for routine use in the management of lung cancer, such as for use in monitoring lung cancer e.g. cancer reoccurrence or response to treatment. Similarly, there is currently no circulating protein biomarker available for routine use for assisting in the diagnosis of lung cancer. The availability of an immunoassay which can be used as a liquid biopsy has the potential to revolutionise the management of cancers, and in particular lung cancer. The present invention aims to provide such a solution. The present invention provides an assay which is cost-effective, easy to use and gives rapid results.

For a confirmed cancer diagnosis, pathology based on a biopsy is recommended. However, in clinical practice, repeating such tissue biopsies during disease progression is often a challenge. Moreover, this invasive sampling procedure is not appropriate for patients during disease progression as it can cause undue health complications, particularly in patients with comorbidities and may further delay treatment administration. Considering these limitations, less invasive and more convenient techniques to monitor treatments in an assay are urgently needed. The present invention seeks to address this need by eliminating or reducing the number of biopsies that are needed over the course of the disease. The clinical potential of the present invention includes examining therapeutic response to treatment, monitoring, and residual disease detection.

Embodiments of the present invention increased the identification of disease progression from around 43% to around 58% over conventional methods, thereby increasing the identification of disease progression by over 15%.

According to one aspect of the invention, there is provided a method of analysing cancer in a subject, comprising:
(i) detecting or measuring the level of trimethylation of lysine 27 on histone H3 (H3K27Me3) of a cell-free nucleosome in a body fluid sample obtained from the subject; and
(ii) analysing circulating tumour DNA (ctDNA) obtained from the subject for a tumour-associated mutation.

The level of H3K27Me3 detected or measured and the ctDNA analysed in the body fluid sample is used to analyse the cancer. Therefore, in one embodiment, the method comprises step (iii) using the level of H3K27Me3 and analysis of ctDNA to analyse the cancer.

According to one aspect of the present invention there is provided a method of analysing cancer in a subject, comprising:
detecting or measuring the level of trimethylation of lysine 27 on histone H3 of a cell-free nucleosome (H3K27Me3) in a body fluid sample obtained from the subject; and
using the level detected or measured in the body fluid sample to analyse the cancer.

The present invention finds particular utility in the lung cancer, and non small cell lung cancer (NSCLC) or small cell lung cancer (SCLC). In a particular embodiment the lung cancer is NSCLC. Examples of NSCLC include adenocarcinoma, squamous cell cancer, large cell carcinoma, adenosquamous carcinoma or sarcomatoid carcinoma. The present invention is particularly useful in analysing adenocarcinoma.

By a method for analysing cancer in a subject we include inter alia for monitoring progression of cancer, monitoring treatment, assessing the requirement for treatment, assessing the requirement for a change in treatment, assessing the requirement for investigation in a subject having cancer or suspected of having cancer, and/or assisting in the diagnosis of a subject having or suspected of having cancer, managing a treatment plan in a subject who is suspected of having cancer, has cancer or who has previously received a cancer diagnosis, for assessing relapse following treatment for cancer, assessing the success of cancer treatment with curative intent and assessing the requirement for treatment of a subject or a change in a subject's treatment.

Minimal residual disease (MRD) is the name given to small numbers of leukaemic cells (cancer cells from the bone marrow) that remain in the person during treatment, or after treatment when the patient is in remission (i.e. patients with no symptoms or signs of disease). However, MRD is the major cause of relapse in cancer and leukaemia. Methods of the invention are therefore useful in monitoring patients who are suspected of relapse, particularly patients who are in remission from cancer.

The selected biomarker comprises the histone post-translational modification (PTM) H3K27Me3 (i.e. tri-methylated histone H3 at the lysine residue in position 27). As the biomarker described herein refers to a histone PTM present in nucleosomes, said biomarker may also be referred to as a “nucleosome biomarker”. Therefore, according to an aspect of the invention, there is provided the use of the nucleosome biomarker H3K27Me3, in a body fluid sample for diagnosing and/or monitoring cancer, particularly lung cancer. In specific aspects, the presence of this biomarker in blood, plasma or serum, for example, is an indicator of lung cancer, although other fluids or tissues may be assayed.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller (2007)).

In one embodiment, the nucleosome is a cell free nucleosome. References to “nucleosome” may refer to “cell free nucleosome” when detected in body fluid samples. It will be appreciated that the term “cell free nucleosome” used throughout this document is intended to include any cell free chromatin fragment that includes one or more nucleosomes.

In one embodiment, the biomarker comprises a histone isoform. Many histone isoforms are known in the art. The nucleotide sequences of a large number of histone isoforms are publicly available for example in the National Human Genome Research Institute NHGRI Histone DataBase (Mariño-Ramírez et al. The Histone Database: an integrated resource for histones and histone fold-containing proteins. Database Vol. 2011), the GenBank (NIH genetic sequence) DataBase, the EMBL Nucleotide Sequence Database and the DNA Data Bank of Japan (DDBJ). In a preferred embodiment, the biomarker comprises a histone isoform of histone H3, for example a histone isoform selected from H3.1, H3.2 and H3t.

In one embodiment assay cut-off levels are used and the patient is considered positive in the test if the assay results are above (or below if applicable) the assay cut-off level for all or a minimum number of the assays (for example, one of two, two of two, two of three etc). In one embodiment of the invention a decision tree model or algorithm is employed for analysis of the results. For example, this assay cut-off level may be greater than or equal to about 10 ng/ml, about 20 ng/ml or about 30 ng/ml. In one embodiment this assay cut-off level is greater than or equal to about 22.5 ng/ml. In a further embodiment, this assay cut-off level is greater than about 22.5 ng/ml.

A substantial proportion of tumour DNA circulates following primary tumour cell necrosis and/or apoptosis and the death of circulating tumour cells. This is often referred to as circulating tumour DNA or ctDNA. In cancer patients, this ctDNA forms a portion of all circulating or cell free DNA known as cfDNA. Following developments in the sensitivity of mutation detection tools, it is now possible to detect some somatic alterations in small plasma samples and this can be used as an aid to cancer patient management.

Several techniques are currently available to detect plasma DNA, including PCR assays and NGS. Of note the limited sensitivity of all methods used for detection in plasma may lead to false-negative results, especially in the case of low cfDNA input. In particular, clinical NGS assays present great opportunities for precision medicine as they can screen thousands of genomic loci for pathogenic alterations. Liquid biopsies are particularly promising, offering a minimally invasive approach to survey ctDNA. However, like many ground-breaking NGS applications, liquid biopsies require detection of low variant allele frequencies and therefore NGS assays may be prone to false negatives. False negatives pose a safety risk and can lead to incorrect treatment decisions or delays while reconfirming results.

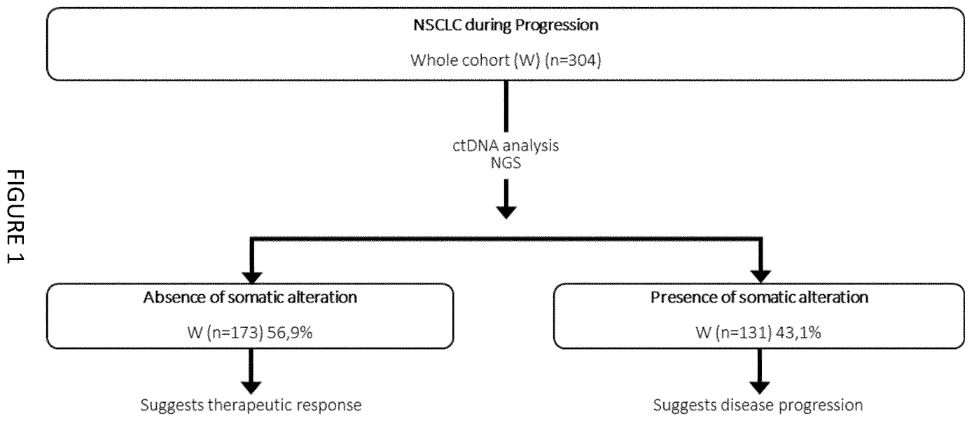

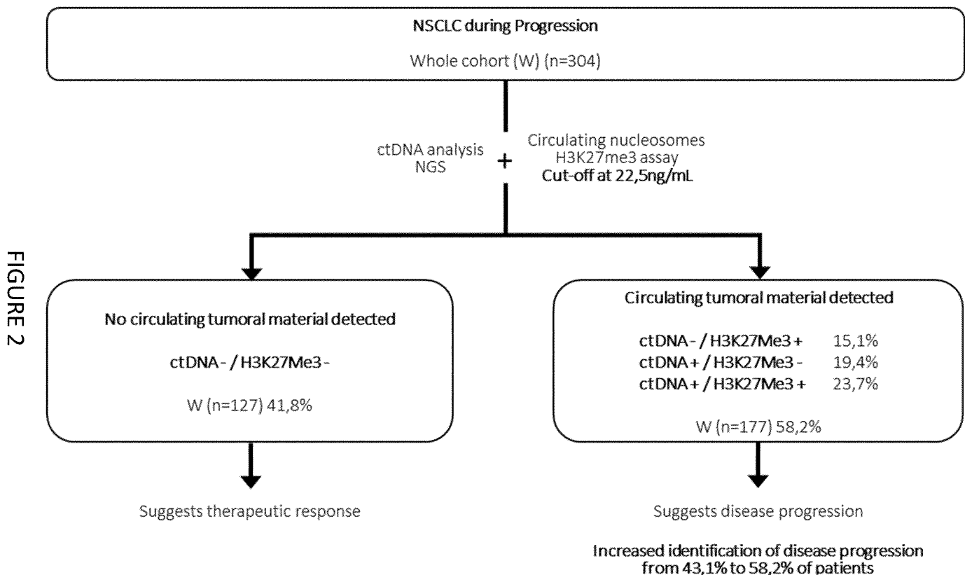

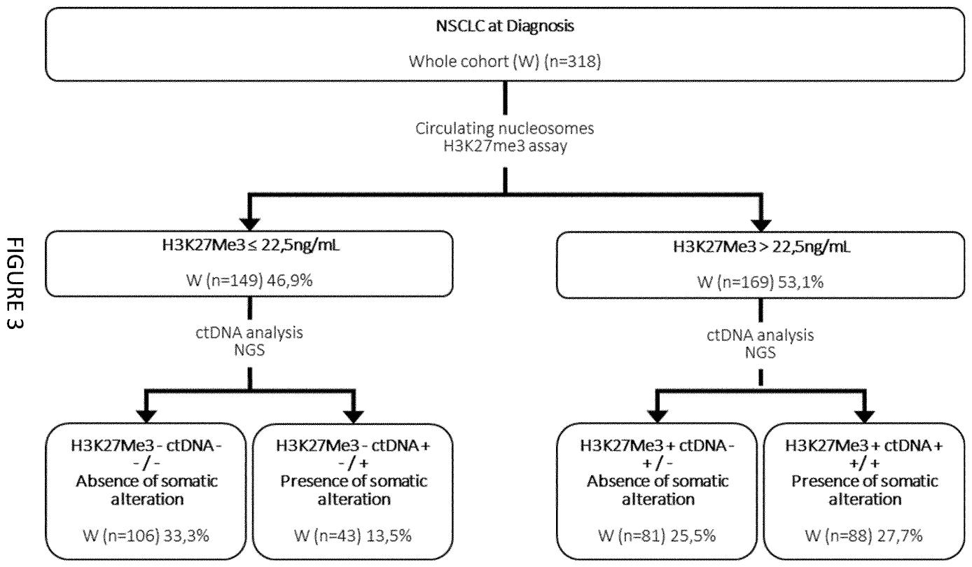

The use of H3K27Me3 according to the invention provides the advantage of reducing such false-negatives. Benefits of the invention are illustrated in FIG. 1 and FIG. 2. FIG. 1 shows that current patient pathway during NSCLC progression after or during treatment. Under this pathway in a cohort of 304 patients the absence of somatic alterations in the cfDNA in 173 patients (or 56.9% of the patients) would suggest a therapeutic response to treatment. The presence of somatic alterations in ctDNA in 131 patients (or 43.1%) would suggest disease progression. The present invention allows for an improved workflow as show in FIG. 2. When the level of H3K27Me3− nucleosomes is also taken into account there is an increased identification of disease progression from 43.1% to 58.2% of patients, i.e., the invention is able to increase the identification of disease progression in this patient cohort. It will be appreciated that this improvement in the identification of disease progression in a liquid biopsy gives rise to substantial benefits, for example allowing early interventions in treatment management.

Therefore in one advantageous embodiment measuring or detecting H3K27Me3 in accordance with the invention is used in combination with ctDNA measurements. In particular the present invention is used in combination with molecular profiling of ctDNA and in particular mutation detection assays. Such mutations may be somatic mutations. ctDNA can be analysed by any convenient method such as PCR or NGS.

Examples of ctDNA analysis techniques which can be used in the present invention are described in:
J. Garcia et al., Evaluation of pre-analytical conditions and comparison of the performance of several digital PCR assays for the detection of major EGFR mutations in circulating DNA from non-small cell lung cancers: the CIRCAN_0 study. Oncotarget 8, 87980-87996 (2017);
J. Bieler et al., High-Throughput Nucleotide Resolution Predictions of Assay Limitations Increase the Reliability and Concordance of Clinical Tests. JCO Clin Cancer Inform 5, 1085-1095 (2021);
J. Garcia et al., Routine Molecular Screening of Patients with Advanced Non-SmallCell Lung Cancer in Circulating Cell-Free DNA at Diagnosis and During Progression Using OncoBEAM™ EGFR V2 and NGS Technologies. Mol Diagn Ther 25, 239-250 (2021); and
J. Garcia et al., Sensitivity, specificity, and accuracy of a liquid biopsy approach utilizing molecular amplification pools. Sci Rep 11, 10761 (2021).

Examples of tumour-associated genes which may be evaluated as part of the present invention include one or more genes selected from the list consisting of: AKT1, ARID1A, BAP1, ARID2, CDK4, CDKN2A, EGFR, FBXW7, FOXL2, IDH2, JAK2, KIT1, APC, ARID2, BRAF, CARD11, CDKN2A, CCND1, DNMT3A, ERBB2, ERBB4, FBXW7, FGFR3, JAK1, KRAS, NRAS, MCL1, MET, NOTCH1, PI3KCA, POLE, PTEN, SF3B1, SMAD4, TP53, ALK, NRG1, EBLN2, LRP1B, EGFR, ELOB, LTBP4, MCM3AP, RET, ESR1, BCL2, GNAQ, RB1 and PDGFR.

Embodiments of the invention use the combination of H3K27Me3 and the presence of ctDNA to improve the accuracy or certainty of diagnosis of lung cancer in clinical settings and enhances the capability to detect lung cancer. For example, as shown in FIG. 3 the data contained herein of the 27.7% of patients who were positive for both H3K27Me3 and the ctDNA at diagnosis, 25.5% of these patients were positive for H3K27Me3 alone showing that H327Me3 is useful as a biomarker at diagnosis.

In some embodiments, the mere presence or absence of a H3K27Me3, without quantifying the amount of this marker, is useful and can be correlated with a diagnosis of lung cancer or increased risk of developing lung cancer.

In other embodiments, the measurement of H3K27Me3 can involve quantifying the marker to correlate the detection of the marker with a probable diagnosis of lung cancer. Thus, if the amount of the marker detected in a subject being tested is different compared to a control amount (i.e., higher than the control), then the subject being tested has a higher probability of having lung cancer.

In certain embodiments of the present invention, the methods comprise managing subject treatment based on their cancer status. As aforesaid, such management includes action subsequent to determining lung cancer status. For example, if the result of the methods of the present invention indicate the presence of disease or progression, the physician may order more investigations. Alternatively, if the status indicates that surgery is appropriate, the patient may be scheduled for surgery. In other instances, the patient may receive chemotherapy or radiation treatments, either in lieu of, or in addition to, surgery. Likewise, if the result is negative, no further action may be warranted. Furthermore, if the results show that treatment has been successful, no further management may be necessary.

These further investigations that may be carried out include an X-ray, CT-scan, PET-scan or biopsy.

One aspect of the present invention concerns the detection of cancer, particularly lung cancer, in an individual or identifying an individual with an increased risk of developing such a cancer. In some embodiments, the invention offers diagnostic benefits for patients with cancer, for example by helping medical care providers diagnose cancer in very early stages that can substantially improve the survival rate in these patients.

In certain embodiments of the invention, there is provided a combination of H3K27Me3 and ctDNA for use as a diagnostic tool for lung cancer (hereinafter “combination of biomarkers”). This combination provides a composite scoring in the assessment and/or diagnosis of cancer. In one embodiment, the level of H3K27Me3 is measured as one of a panel of measurements, such as including DNA analysis. In a further embodiment, the DNA is analysed for circulating tumour DNA (ctDNA). In an even further embodiment, the ctDNA is analysed for a tumour-associated mutation such as a tumour-associated somatic mutation.

Therefore, according to one aspect of the present invention there is provided a method of analysing cancer in a subject, comprising:
(i) detecting or measuring the level of trimethylation of lysine 27 on histone H3 (H3K27Me3) of a cell-free nucleosome in a body fluid sample obtained from the subject; and
(ii) analysing ctDNA obtained from the subject for a tumour-associated mutation such as a tumour-associated somatic mutation.

In some embodiments, the method additionally comprises using the H3K27Me3 levels detected or measured in the body fluid sample in combination with the analysis of the ctDNA to analyse the cancer.

In some embodiments, the presence of ctDNA in combination with a higher level of H3K27Me3 compared to a control is indicative of the presence and/or progression of cancer, is indicative that the subject requires further investigation, such as a lung biopsy, and/or is indicative that the subject requires treatment or a change of treatment.

In some embodiments, the absence of ctDNA in combination with a higher level of H3K27Me3 compared to a control is indicative of the presence and/or progression of cancer, is indicative that the subject requires further investigation, such as a lung biopsy, and/or is indicative that the subject requires treatment or a change of treatment.

In some embodiments, the absence of ctDNA in combination with a lower level of H3K27Me3 compared to a control is indicative of the absence and/or regression of cancer (e.g. during treatment), is indicative that the subject does not require further investigation, and/or is indicative that the subject does not require treatment or is successfully responding to treatment.

According to a further aspect, there is provided a method of monitoring cancer progression in a subject (e.g. a subject with cancer, such as lung cancer), comprising:
(i) detecting or measuring the level of H3K27Me3 of a cell-free nucleosome in a body fluid sample obtained from the subject; and
(ii) analysing ctDNA obtained from the subject for a tumour-associated mutation such as a tumour-associated somatic mutation,
   wherein the level of H3K27Me3 and/or the presence of a tumour-associated mutation in the ctDNA is indicative of the progression of the disease (e.g. cancer).

According to a further aspect, there is provided a method of diagnosing cancer (e.g. lung cancer) in a subject, comprising:
(i) detecting or measuring the level of H3K27Me3 of a cell-free nucleosome in a body fluid sample obtained from the subject; and
(ii) analysing ctDNA obtained from the subject for a tumour-associated mutation such as a tumour-associated somatic mutation,
   wherein the level of H3K27Me3 and/or the presence of a tumour-associated mutation in the ctDNA is indicative that the subject has cancer.

As described herein, if the method indicates that the subject has cancer, they may be subsequently investigated using further diagnostic investigations, such as biopsy, to confirm the diagnosis. In one embodiment, the presence of ctDNA in combination with a higher level of H3K27Me3 compared to a control is indicative that the subject requires further investigation, such as a lung biopsy.

The invention also provides for such methods where the biomarker (or combination of biomarkers) are measured again after subject management. In these cases, the methods are used to monitor the status of the cancer, e.g., response to cancer treatment, remission of the disease or progression of the disease. Because of the ease of use of the methods and the lack of invasiveness at least in certain embodiments of the methods, the methods can be repeated after each treatment the patient receives. This allows the effectiveness of the course of treatment to be followed. If the results show that the treatment is not effective, the course of treatment can be altered accordingly. This enables flexibility in treatment options.

In one embodiment the treatment is selected from: chemotherapy, radiotherapy (including stereotactic ablative radiotherapy (SABR) or stereotactic body radiation therapy (SBRT), and continuous hyperfractionated accelerated radiotherapy (CHART)), chemoradiation, surgery, targeted cancer drugs, immunotherapy, radiofrequency ablation, photodynamic therapy and/or symptom control treatment. The treatment is a second line treatment.

In one embodiment of the invention a positive result may indicate that treatment targeting EZH2 is appropriate, such as treatment with an EZH2 inhibitor.

In other particular embodiments the present invention provides:

A method of treating lung cancer and/or assessing lung cancer comprising: obtaining a body fluid sample from a subject;
(i) measuring the level of H3K27Me3 in the sample;
(ii) identifying the subject as suffering from lung cancer based on a higher level of H3K27Me3 compared to a control; and
(iii) administering a treatment to the subject.

A method for monitoring the success of lung cancer treatment with curative intent comprising detecting levels of H3K27Me3 in a body fluid sample from a subject undergoing treatment for lung cancer for comparison with the previous level obtained from the subject.

A method for monitoring the progression of lung cancer in a subject comprising:
(a) determining a level of a H3K27Me3 4 in a body fluid sample obtained from a subject;
(b) optionally comparing the amount of the determined H3K27Me3 in the sample from the subject to the amount of the H3K27Me3 in a normal control; and
(c) repeating steps (a) and (b) at two or more time intervals, wherein an increase in the amount of the H3K27Me3 level from the patient over time is associated with an increase in the progression of lung cancer and a decrease in the amount of the H3K27Me3 level from the patient over time is associated with a decrease in the progression of lung cancer.

A method for predicting the clinical outcome of a patient suffering of lung cancer, comprising the steps of: a) assessing the level of H3K27Me3 in a biological sample of the patient; and b) comparing the level of H3K27Me3 to a reference value; c) predicting the clinical outcome on the basis of the comparison of step b).

A method of assessing activity of an anti-tumoral treatment in a lung cancer patient treated with the anti-tumoral treatment, said method comprising:
(a) measuring the level of H3K27Me3 in a body fluid sample, preferably a plasma sample, from the cancer patient prior to administering the anti-tumoral treatment; and
(b) measuring the level of H3K27Me3 in a biological sample, preferably a plasma sample, from the cancer patient, wherein a level of methylation which is decreased as compared to the level of H3K27Me3 prior to administering the anti-tumoral treatment to the lung cancer patient indicates activity of anti-tumoral treatment in the lung cancer patient, such as wherein the activity is dose-dependent activity.

These aforementioned embodiments can in one preferred embodiment be carried out using the combination of H3K27Me3 and ctDNA analysis as biomarkers.

The methods of the present invention have other applications as well. For example, the marker can be used to screen for compounds that modulate the expression of the marker in vitro or in vivo, which compounds in turn may be useful in treating or preventing lung cancer in patients. In another example, the marker can be used to monitor the response to treatments for lung cancer. In yet another example, the marker can be used in heredity studies to determine if the subject is at risk for developing lung cancer. For instance, certain markers may be genetically linked. This can be determined by, e.g., analysing samples from a population of lung cancer patients whose families have a history of lung cancer. The results can then be compared with data obtained from, e.g., lung cancer patients whose families do not have a history of lung cancer. The marker that are genetically linked may be used as a tool to determine if a subject whose family has a history of lung cancer is pre-disposed to having lung cancer.

As used herein, the term “biomarker” refers to a marker that allows detection of disease or that can assist in the detection of disease in an individual when compared to a healthy individual, including detection of residual disease, disease reoccurrence and disease in its early stages. In specific embodiments, the level of the biomarker as determined by immunoassay in fluid samples from an individual to be tested is compared with respective levels in previous fluid samples from the same individual or from fluid samples from another healthy individual.

The sample may be any biological fluid (or body fluid) sample taken from a subject including, without limitation, cerebrospinal fluid (CSF), whole blood, blood serum, plasma, menstrual blood, endometrial fluid, urine, saliva, or other bodily fluid (stool, tear fluid, synovial fluid, sputum), breath, e.g. as condensed breath, or an extract or purification therefrom, or dilution thereof. In a preferred embodiment, the body fluid sample is selected from blood, serum or plasma. Biological samples also include specimens from a live subject, or taken post-mortem. The samples can be prepared, for example where appropriate diluted or concentrated, and stored in the usual manner. It will be understood that methods and uses of the present invention find particular use in blood, serum or plasma samples obtained from a patient. In one embodiment, the sample is a blood or plasma sample. In a further embodiment, the sample is a serum sample. In a further embodiment both serum and plasma samples are used for the measurement of different members of an assay panel.

In one embodiment, the biomarkers are for use in diagnosing the stage of cancer. Cancer may be assigned as stage 0, stage I, stage II, stage III and stage IV. Stage definition varies with different cancer diseases and is known in the art. Typically, stage I is classified as when the cancer is small and confined locally to the tissue of origin. Stage II is classified as when the cancer has grown larger and beyond its origin into nearby tissues within the organ or to nearby lymph nodes. Stage III is classified as when the cancer has grown into nearby tissues beyond the organ of origin but has not spread to other more distant parts of the body. Stage IV is classified as when the cancer has spread to one or more distant parts of the body, such as the liver or lungs. Early stage cancer generally includes stages 0, I and II. Late stage cancer generally includes stages III and IV.

More particularly in NSCLC cancer staging is carried out using a system called TNM, where:
T describes the size of the tumour (cancerous tissue)
N describes the spread of the cancer into lymph nodes
M describes whether the cancer has spread to another area of the body such as the liver (metastasis).

### T

There are 4 main stages for T:
T1 lung cancer means that the cancer is still inside the lung. T1 is broken down into 3 sub-stages:
T1a—the tumour is no wider than 1 cm;
T1b—the tumour is between 1 cm and 2 cm wide; and
T1c—the tumour is between 2 cm and 3 cm wide.

T2 is used to describe 3 possibilities:
the tumour is between 3 cm and 5 cm wide, or
the tumour has spread into the main airway or the inner lining of the chest wall, or
the lung has collapsed or is blocked due to inflammation.

T3 is used to describe 3 possibilities:
the tumour is between 5 cm and 7 cm wide, or
there is more than 1 tumour in the lung lobe, or
the tumour has spread into the chest wall, the phrenic nerve (a nerve close to the lungs), or the outer layer of the heart (pericardium).

T4 is used to describe a range of possibilities including:
the tumour is wider than 7 cm, or
the tumour has spread into both sections of the lung (each lung is made up of 2 sections, known as lobes), or
the tumour has spread into an area of the body near to the lung, such as the heart, the windpipe, the food pipe (oesophagus) or a major blood vessel.

### N

There are 3 main stages for N:
N1 is used to describe cancerous cells in the lymph nodes located inside the lung or in the area where the lungs connect to the airway (the hilum).

N2 is used to describe 2 possibilities:
there are cancerous cells in the lymph nodes located in the centre of the chest on the same side as the affected lung, or
there are cancerous cells in the lymph nodes underneath the windpipe.

N3 is used to describe 3 possibilities:
there are cancerous cells in the lymph nodes located on the chest wall on the other side of the affected lung, or
there are cancerous cells in the lymph nodes above the collar bone, or
there are cancerous cells in the lymph nodes at the top of the lung.

### M

There are 2 main stages for M:
M0'the cancer has not spread outside the lung to another part of the body; and
M1—the cancer has spread outside the lung to another part of the body.

Small-cell lung cancer is less common than non-small-cell lung cancer. The cancerous cells are smaller in size than the cells that cause non-small-cell lung cancer.

Small-cell lung cancer only has 2 possible stages:
limited disease—where the cancer is only in 1 lung and may be in nearby lymph nodes; or
extensive disease—where the cancer has spread to the other lung, to lymph nodes that are further away, or to other parts of your body

### Measurement Methods

In one embodiment, the level or concentration of the H3K27Me3 nucleosome biomarker detected is compared to a control. It will be clear to those skilled in the art that the control subjects may be selected on a variety of basis which may include, for example, subjects known to be free of the disease or may be subjects with a different disease (for example, for the investigation of differential diagnosis). The “control” may comprise a healthy subject, a non-diseased subject and/or a subject without cancer. Comparison with a control is well known in the field of diagnostics.

It will be understood that it is not necessary to measure healthy/non-diseased controls for comparative purposes on every occasion because once the ‘normal range’ is established it can be used as a benchmark for all subsequent tests. A normal range can be established by obtaining samples from multiple control subjects without cancer and testing for the level of biomarker. Results (i.e. biomarker levels) for subjects suspected to have cancer can then be examined to see if they fall within, or outside of, the respective normal range. Use of a ‘normal range’is standard practice for the detection of disease.

If a subject is determined to not have cancer, then the invention may still be used for the purposes of monitoring disease progression. For example, if the use comprises a blood, serum or plasma sample from a subject determined not to have cancer, then the biomarker level measurements can be repeated at another time point to establish if the biomarker level has changed.

References to “subject” or “patient” are used interchangeably herein. In one embodiment, the patient is a human patient. In one embodiment, the patient is a (non-human) animal. The use, panels and methods described herein are preferably performed in vitro.

In one embodiment, detection or measurement of said biomarker comprise an immunoassay, immunochemical, mass spectroscopy, chromatographic, chromatin immunoprecipitation or biosensor method.

In one embodiment, the detection or measurement comprises an immunoassay. In a preferred embodiment of the invention there is provided a 2-site immunoassay method for nucleosome moieties. In particular, such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. In another embodiment of the invention, there is provided a 2-site immunoassay employing a labelled anti-nucleosome detection binding agent in combination with an immobilized anti-histone modification or anti-histone variant or anti-DNA modification or anti-adducted protein binding agent.

Detecting or measuring the level of the biomarker may be performed using one or more reagents, such as a suitable binding agent. In one embodiment, the one or more binding agents comprises a ligand or binder specific for the desired H3K27Me3 biomarker, or a structural/shape mimic of the biomarker or component part thereof.

It will be clear to those skilled in the art that the terms “antibody”, “binder” or “ligand” in regard to any aspect of the invention is not limiting but intended to include any binder capable of binding to particular molecules or entities and that any suitable binder can be used in the method of the invention.

Methods of detecting biomarkers are known in the art. In one embodiment, the reagents comprise one or more ligands or binders. In one embodiment, the ligands or binders of the invention include naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

A further aspect of the invention is a kit for detecting the presence of a disease state, comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace. Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers of the invention.

Biomarkers for detecting the presence of a disease are essential targets for discovery of novel targets and drug molecules that retard or halt progression of the disorder. As the result for a biomarker or biomarker panel is indicative of disorder and of drug response, the biomarker is useful for identification of novel therapeutic compounds in in vitro and/or in vivo assays. Biomarkers and biomarker panels of the invention can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying subjects most likely to respond to a particular therapeutic treatment, drug screening and development. Biomarkers and uses thereof are valuable for identification of new drug treatments and for discovery of new targets for drug treatment.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

Identifying and/or quantifying can be performed by any method suitable to identify the presence and/or amount of a specific protein in a biological sample from a subject or a purification or extract of a biological sample or a dilution thereof. In methods of the invention, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner.

Identification and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed using an immunological method, such as an immunoassay. Immunoassays include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels.

In another example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving identification and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

The identification of biomarkers for a disease state permits integration of diagnostic procedures and therapeutic regimes. Detection of a biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus, by monitoring a biomarker of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

### Kits and Panel Tests

The combination of markers described herein may be used to prepare a kit or panel test, in particular for use in the diagnosis of lung cancer and/or monitoring of patients with lung cancer or suspected lung cancer.

Therefore, according to a further aspect of the invention, there is provided a kit comprising reagents to detect the level of H3K27Me3. According to a further aspect of the invention, there is provided a kit comprising reagents to detect H3K27Me3 and ctDNA. For the detection of H3K27Me3 (in particular, when associated with circulating nucleosomes), the reagents may comprise a binding agent which specifically binds H3K27Me3, and optionally one or more additional binding agents which specifically bind nucleosomes or a further epigenetic feature thereof. For the detection of cfDNA, the reagents may comprise reagents for DNA extraction and/or sequencing. The kits described herein may be for use in the diagnosis of lung cancer.

According to a further aspect of the invention there is provided the use of the kit as defined herein to identify a patient in need of treatment for lung cancer.

According to a further aspect of the invention there is provided the use of the kit as defined herein to monitor a patient for progression of lung cancer (e.g. further growth of the tumour, or advancement to a different stage of cancer). Embodiments of this aspect include use to detect disease progression in watchful waiting, active surveillance and monitoring post-surgery or other treatment for relapse.

According to a further aspect of the invention there is provided the use of the kit as defined herein to evaluate the effectiveness of a lung cancer treatment in a patient.

According to a further aspect of the invention there is provided the use of the kit as defined herein to select a treatment for a patient with lung cancer.

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLES

### Methods and Materials

cfDNA Collection

Total EDTA blood samples were centrifuged for 10 min at 1600 g. The supernatant was then centrifuged at 6000 g for 10 min, and the resulting plasma was stored at −80° C. until cfDNA extraction and molecular analyses. CfDNA was extracted using the QIAamp Circulating Nucleic Acid Kit (Qiagen, Valencia, CA, USA, Cat No 55114), with a Qiagen vacuum manifold following the manufacturers' instructions. CfDNA samples were quantified using a Qubit™ 4 Fluorometer (Invitrogen™, Cat No Q33238, Carlsbad CA, 92008, United States) with the Qubit™ dsDNA HS Assay Kit (Invitrogen™, Cat No 32854).

### Library Preparation for DNA Sequencing

For custom-validated NGS library preparation, 10-100 ng cfDNA were used, using a custom capture-based technology provided by SOPHIA GENECTICS (Lausanne, Switzerland) and performed according to the manufacturer's instructions. The custom panel covered 78 genes involved in cancer (such as EGFR, TP53 or KRAS). The libraries were sequenced on NextSeq 550 (Illumina technology, San Diego, CA 92122) in 2×150 paired-end runs. The subsequent Variant Call Files were subjected to cross-sample background filtering, with potential artefacts removed below 3 standard deviations of the mean background noise for each position. Filter criteria for variant calling were set to an absolute number of mutated allele read counts ≥40, a minimal total depth>1000×, and a MAF threshold ≥0.5%. The bioinformatics was performed using SophiaDDM platform.

### H3K27Me3 ImmunoAssay:

Nucleosome structure was measured using a sandwichimmunoassay. based on magnetic beads and chemiluminescence technology. 50 μL of K2-EDTA plasma (same as for the DNA sequencing analysis) was incubated with acridinium ester labeled anti-nucleosome antibody. Then, magnetic particle beads coated with the corresponding monoclonal anti-histone H3K27Me3 capture antibody was added. Finally, after a wash step, a chemiluminescent substrate was added and the light emitted by the acridinium ester was measured by the luminometer system. The results are expressed in relative light unit (RLU) and the concentrations were extrapolated using a four-parameter logistic regression of a reference standard curve. All samples were analyzed in duplicate.

### Results

### Circulating H3K27Me3− Nucleosomes in NSCLC at Diagnosis

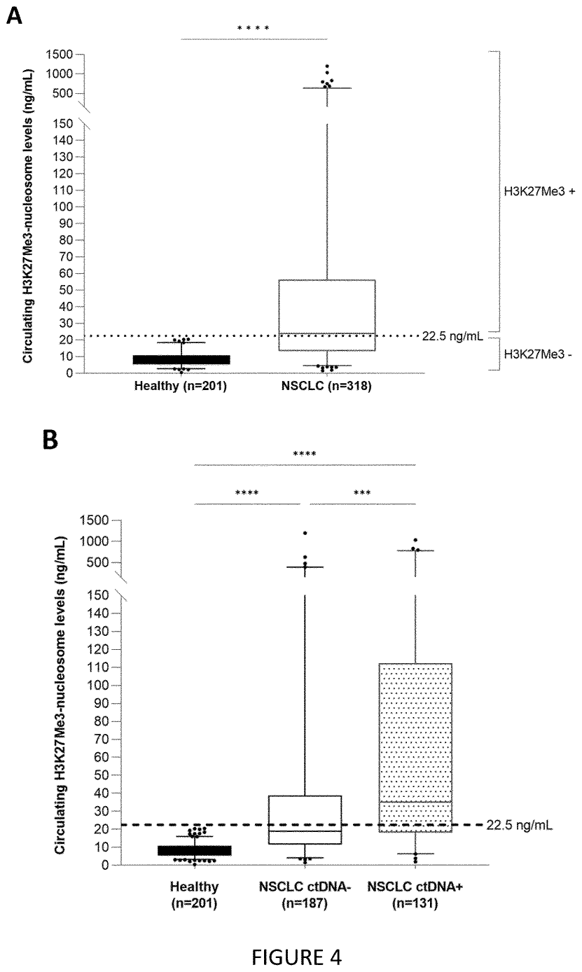

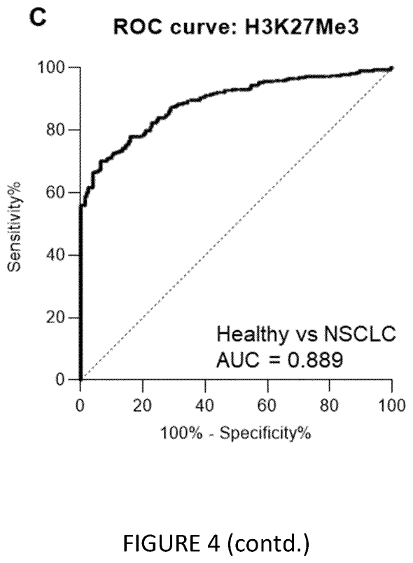

The results of the NSCLC cohort at diagnosis are shown in FIG. 4. In the whole NSCLC cohort (n=318), 46.9% of the patients (n=149) showed a H 3K27Me3− nucleosome level below the cut-off and 53.1% (n=169) are above (FIG. 4A). In the whole NSCLC cohort, there were 41.2% samples for which at least one mutation or copy number variation (CNV) was identified in the NGS panel evaluated, referred as ctDNA positive sample (ctDNA+; n=131) hereafter and 58.8% of patients for whom no genetic alteration was found in their blood samples. They are defined as ctDNA negative (ctDNA−; n=187) hereafter. The level of H3K27Me3− nucleosome is statistically significantly higher in ctDNA+ group compared to ctDNA− group (median=33.9 ng/mL vs 18.5 ng/mL; p-value <0.001) and compared to healthy groups (median=33.9 ng/mL vs 8.0 ng/ml; p-value <0.0001) (FIG. 4B). H3K27Me3− nucleosomes levels in ctDNA− group remained also higher compared to healthy group (median=18.5 ng/ml vs 8.0 ng/ml; p-value <0.0001).

High clinical performances were obtained by ROC curve analysis, with an area under the curve (AUC) of about 0.89.

In summary, circulating H3K27Me3− nucleosome levels are significantly increased in NSCLC samples at diagnosis, even more so when a somatic mutation is detected (ctDNA+).

Based on these observations, the mutational tumor burden defined by the ctDNA status of the samples and the H3K27Me3− nucleosome concentrations was compared (FIG. 3). It was observed that: i) 33.3% of samples were double negative for the ctDNA and H 3K27me3, meaning an absence of tumoral material containing ctDNA or epigenetic biomarkers released or decrease of expression of the H 3K27Me3 in the tissue tumor ; ii) 13.5% samples had low level of H3K27Me3, even if somatic alterations have been detected, indicating decreased tumor epigenetic remodelling processes in these NSCLC cancers, iii) 25.5% of samples were negative for somatic alteration but had a high level of H3K27me3− nucleosomes, indicative of the presence of tumoral epigenetic process; iv) 27.7% samples were found positive for both ctDNA and H3K27me3− nucleosomes, pointing to both maintenance epigenetic and mutational burden processes.

### Circulating H3K27Me3− Nucleosomes in NSCLC During Progression

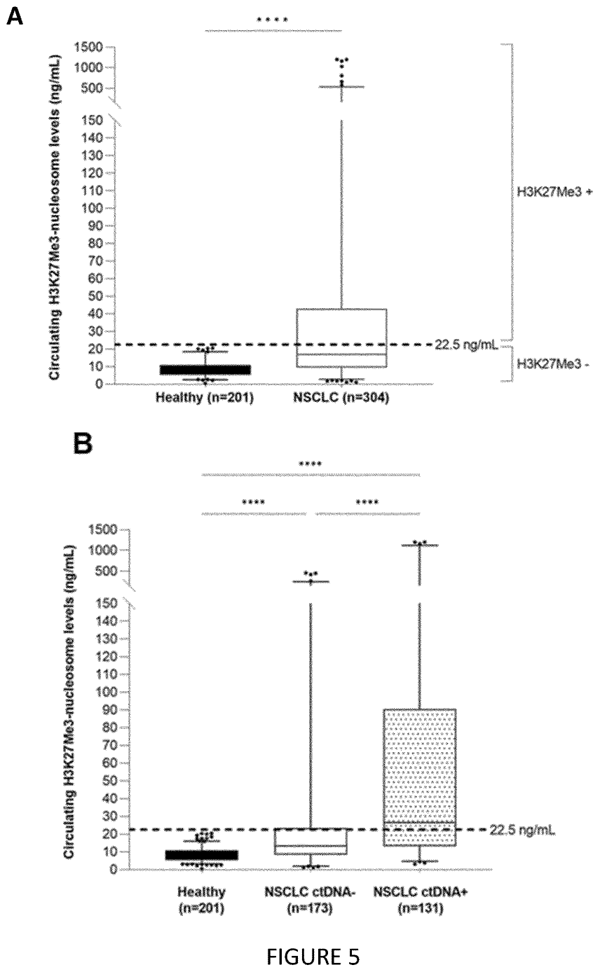

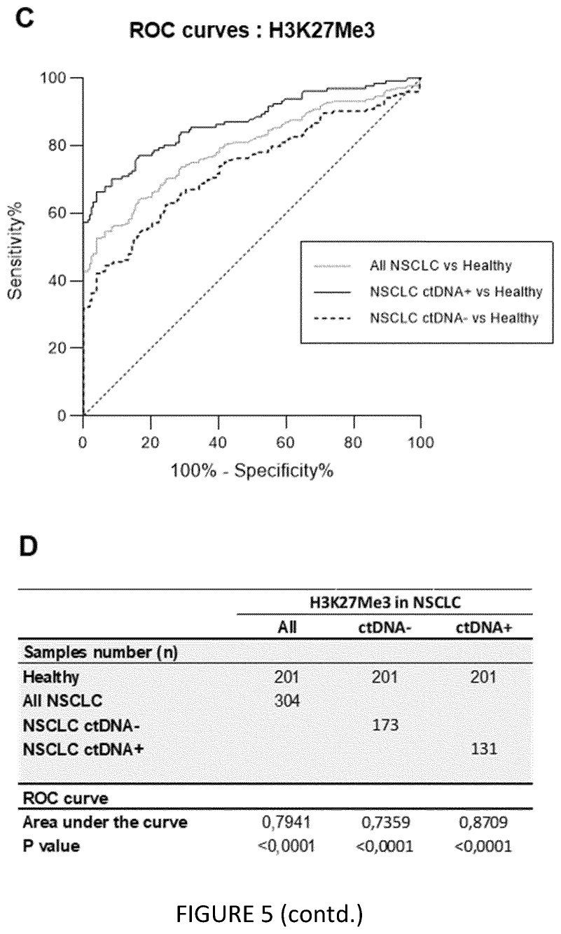

The results of the NSCLC cohort during progression are shown in FIG. 5. The concentration of circulating H3K27Me3− nucleosomes was measured in NSCLC samples collected during treatment progression (n=304) and compared to healthy samples (n=201), in routine use conditions. A highly significant increase is observed in NSCLC compared to healthy samples (median=16.89 ng/mL vs. 7.99 ng/ml, respectively; p-value <0.0001) (FIG. 5A). In this NSCLC population during treatment, the H3K27Me3− nucleosome levels were lower to the one detected in the NSCLC samples at diagnosis (median_{DURING PROGRESSION}=16.89 ng/ml vs. median_{AT DIAGNOSIS}=24.01 ng/ml).

The molecular profiles of the NSCLC samples collected during treatment were conducted by NGS analyses. In 43.1% of the samples (n=131), at least one mutation among the 78 screened was detected, confirming the presence of ctDNA in plasma samples. We then compared the circulating H3K27Me3− nucleosome levels in positive (ctDNA+) and negative (ctDNA−) mutated ctDNA samples collected during treatment progression. Circulating H3K27Me3− nucleosomes level is lower in the ctDNA− negative group compared to ctDNA− positive group (median_{ctDNA-}=13.35 ng/ml vs median_{ctDNA+}=26.05 ng/ML, respectively, p_value<0.0001) (FIG. 5B). As described previously, a cut-off at 22.5 ng/ml of H 3K27Me3− nucleosomes was determined. Based on this cut-off, the NSCLC samples collected during treatment progression can be classified as H3K27Me3−negative (H3K27Me− below or equal to 22.5 ng/mL) and H3K27Me3− positive (H3K27Me3+ above 22.5 ng/ml). Most of the ctDNA− samples are also H3K27Me3−, represented by the upper limit of the box plot close to the cut-off (FIG. 5B), whereas the majority of the ctDNA+ samples are also H3K27Me3+, shown by a median above 22.5 ng/mL.

A higher range in circulating H3K27Me3− nucleosomes is observed in ctDNA+group compared to ctDNA− and healthy groups (ctDNA+ samples minimum 3.11 ng/mL and maximum over 1200 ng/ml); ctDNA− samples minimum 1.15 ng/ml and maximum 456.10 ng/ml maximum values; healthy samples minimum 0.47 ng/mL and maximum 20.51 ng/ml values).

The clinical performances were evaluated on the whole cohort, on the ctDNA− and ctDNA+ sub-groups in comparison to the healthy samples (FIG. 5C-D). The discrimination of NSCLC samples is better in ctDNA+ sub-group with an area under the curves about 0.87 compared to 0.74 for ctDNA− sub-group and 0.79 for the whole cohort respectively; p_value<0.0001).

In summary, circulating H3K27Me3− nucleosome levels are significantly increased in NSCLC samples during progression, even more so when somatic mutation is detected (ctDNA+). After sub-grouping of NSCLC samples into ctDNA− and ctDNA+ classification; samples were classified regarding the level of circulating H3K27Me3− nucleosomes and finally organized in four classes described in a decision tree (FIG. 2). 41.8% of samples were double negative for the ctDNA and circulating H3K27Me3− nucleosome levels. 15.1% of samples that are negative for somatic alteration had a high concentration of circulating H3K27Me3− nucleosome levels. 19.4% of samples had weak level of circulating H3K27Me3− nucleosome, even when somatic alterations had been detected. 23.68% samples were found positive for both ctDNA and circulating H3K27Me3− nucleosome levels.

### CLAUSES

A set of clauses defining the invention and its preferred aspects is as follows:

Clause 1. A method of analysing cancer in a subject, comprising:
detecting or measuring the level of trimethylation of lysine 27 on histone H3 (H3K27Me3) of a cell-free nucleosome in a body fluid sample obtained from the subject; and using the level detected or measuring in the body fluid sample to analyse the cancer.

Clause 2. The method of clause 1, wherein the cancer is lung cancer.

Clause 3. The method of clause 2, wherein the lung cancer is non small cell lung cancer (NSCLC) or small cell lung cancer (SCLC).

Clause 4. The method of clause 3, wherein the lung cancer is NSCLC.

Clause 5. The method of clause 4, wherein the NSCLC is adenocarcinoma, squamous cell cancer, large cell carcinoma, adenosquamous carcinoma or sarcomatoid carcinoma.

Clause 6. The method of clause 5, wherein the NSCLC is adenocarcinoma.

Clause 7. The method of any preceding clause for monitoring progression of cancer, monitoring treatment, assessing the requirement for treatment, assessing the requirement for a change in treatment, assessing the requirement for investigation in a subject having cancer or suspected of having cancer, and/or assisting in the diagnosis of a subject having or suspected of having cancer.

Clause 8. The method of any preceding clause for managing a treatment plan in a subject who is suspected of having cancer, has cancer or who has previously received a cancer diagnosis.

Clause 9. The method of any preceding clause for assessing relapse following treatment for cancer.

Clause 10. The method of clause 9, wherein the subject has minimal residual disease (MRD).

Clause 11. The method of any preceding clause for assessing the success of cancer treatment with curative intent.

Clause 12. The method of any preceding clause for assessing the requirement for treatment of a subject or a change in a subject's treatment.

Clause 13. The method of any preceding clause wherein the level of H3K27Me3 is compared to a control or a previous level obtained from the subject.

Clause 14. The method of any preceding clause for monitoring a change in stage of cancer, wherein an increase, relative to an earlier stage sample or control is indicative of progression of the cancer from an earlier stage to later stage of disease.

Clause 15. The method of any preceding clause wherein the subject is subsequently investigated for cancer and the investigation includes pulmonary function test (PFT), imaging, biopsy and/or surgery.

Clause 16. The method of clause 15, wherein investigation is a biopsy.

Clause 17. The method of clause 15, where the imaging is an x-ray, a chest computed tomography (CT) scan, or a positron emission tomography (PET) scan.

Clause 18. The method according to any preceding clause, wherein the level of H3K27Me3 which is indicative of the presence and/or progression of cancer is greater than or equal to about 22.5 ng/ml.

Clause 19. The method of any preceding clause, wherein the level of H3K27Me3 is measured as one of a panel of measurements including DNA analysis.

Clause 20. The method of clause 19, wherein the DNA is analysed for circulating tumour DNA (ctDNA).

Clause 21. The method of clause 20, wherein the ctDNA is analysed for a tumour-associated mutation such as a tumour-associated somatic mutation.

Clause 22. The method according to any one of clauses 19 to 21, wherein the presence of ctDNA in combination with a higher level of H3K27Me3 compared to a control is indicative of the presence and/or progression of cancer, is indicative that the subject requires further investigation, such as a lung biopsy, and/or is indicative that the subject requires treatment or a change of treatment.

Clause 23. The method according to any one of clauses 19 to 21, wherein the absence of ctDNA in combination with a higher level of H3K27Me3 compared to a control is indicative of the presence and/or progression of cancer, is indicative that the subject requires further investigation, such as a lung biopsy, and/or is indicative that the subject requires treatment or a change of treatment.

Clause 24. The method of any preceding clause, wherein the cancer is stage I, II, III or IV.

Clause 25. The method of any preceding clause, wherein the cancer is described as TX, T0, Tis, T1 (including T1mi, T1a, T1b, T1c), T2 (including T2a, T2b), T3 or T4; wherein the cancer is described as NX, N0, N1, N2 or N3 and/or wherein the cancer is described as M0 or M1 (including M1a, M1b, M1c).

Clause 26. A method of analysing cancer in a subject, comprising:
(i) detecting or measuring the level of trimethylation of lysine 27 on histone H3 (H3K27Me3) of a cell-free nucleosome in a body fluid sample obtained from the subject; and
(ii) analysing ctDNA obtained from the subject for a tumour-associated mutation such as a tumour-associated somatic mutation.

Clause 27. Use of H3K27Me3 as a biomarker in a body fluid sample for monitoring progression of cancer, monitoring treatment, assessing the requirement for treatment, assessing the requirement for a change in treatment, assessing the requirement for investigation in a subject having cancer or suspected of having cancer, or assisting in the diagnosis of a subject having or suspected of having cancer.

Clause 28. A kit comprising reagents to detect H3K27Me3 and optionally one or more biomarkers including ctDNA.

1. A method of analysing cancer in a subject, comprising:
(i) detecting or measuring the level of trimethylation of lysine 27 on histone H3 (H3K27Me3) of a cell-free nucleosome in a body fluid sample obtained from the subject; and
(ii) analysing circulating tumour DNA (ctDNA) obtained from the subject for a tumour-associated mutation.

2. The method according to claim 1, wherein:
(i) the presence of ctDNA in combination with a higher level of H3K27Me3 compared to a control is indicative of the presence and/or progression of cancer, is indicative that the subject requires further investigation, and/or is indicative that the subject requires treatment or a change of treatment; or
(ii) wherein the absence of ctDNA in combination with a higher level of H3K27Me3 compared to a control is indicative of the presence and/or progression of cancer, is indicative that the subject requires further investigation, and/or is indicative that the subject requires treatment or a change of treatment.

3. The method of claim 1, wherein the cancer is lung cancer.

4. The method of claim 3, wherein the lung cancer is non small cell lung cancer (NSCLC) or small cell lung cancer (SCLC).

5. The method of claim 4, wherein the lung cancer is NSCLC.

6. The method of claim 5, wherein the NSCLC is adenocarcinoma, squamous cell cancer, large cell carcinoma, adenosquamous carcinoma or sarcomatoid carcinoma.

7. (canceled)

8. The method of claim 1, wherein the tumour-associated mutation is a tumour-associated somatic mutation.

9. (canceled)

10. The method of claim 1, wherein the method is for assessing relapse following treatment for cancer.

11. The method of claim 1, wherein the subject has minimal residual disease (MRD).

12. The method of claim 1, wherein the method is for assessing the success of cancer treatment with curative intent and/or assessing the requirement for treatment of a subject or a change in a subject's treatment.

13. The method of claim 1, wherein the level of H3K27Me3 is compared to a control or a previous level obtained from the subject.

14. The method of claim 1, wherein the method is for monitoring a change in stage of cancer, wherein an increase, relative to an earlier stage sample or control is indicative of progression of the cancer from an earlier stage to later stage of disease.

15. The method of claim 1, further comprising subsequently investigating the subject for cancer, wherein the investigating comprises a pulmonary function test (PFT), imaging, biopsy and/or surgery.

16. (canceled)

17. The method of claim 15, where the imaging is an x-ray, a chest computed tomography (CT) scan, or a positron emission tomography (PET) scan.

18. The method of claim 1, wherein the level of H3K27Me3 which is indicative of the presence and/or progression of cancer is greater than or equal to about 22.5 ng/ml.

19. The method of claim 1, wherein the cancer is stage I, II, III or IV.

20. The method of claim 1, wherein the cancer is described as TX, T0, Tis, T1 (including T1mi, T1a, T1b, T1c), T2 (including T2a, T2b), T3 or T4, or wherein the cancer is described as NX, N0, N1, N2 or N3, or wherein the cancer is described as M0 or M1 (including M1a, M1b, M1c).

21. The method of claim 1, wherein the level of H3K27Me3 is measured as one of a panel of measurements.

22. (canceled)

23. A kit comprising reagents to detect H3K27Me3 and ctDNA.
